(19) United States
(12) Patent Application Publication
(10) Pub. No.: US 2022/0218465 A1
(43) Pub. Date: Jul. 14, 2022

(54) MODIFIED EXTRACELLULAR MATRIX-BASED HYDROGEL, MANUFACTURING METHOD OF THE SAME AND USE OF THE SAME

(71) Applicant: POSTECH RESEARCH AND BUSINESS DEVELOPMENT FOUNDATION, Pohang-si (KR)

(72) Inventors: Hyeon Ji KIM, Changwon-si (KR); Dong Woo CHO, Seoul (KR); Jin Ah JANG, Pohang-si (KR)

(21) Appl. No.: 17/502,152

(22) Filed: Oct. 15, 2021

(30) Foreign Application Priority Data
Jan. 14, 2021 (KR) 10-2021-0005008

(51) Int. Cl. A61F 2/14; A61L 27/36; A61L 27/38; B29C 64/106

(52) U.S. Cl. CPC A61F 2/142; A61L 27/3633; A61L 27/3675; B33Y 80/00; A61L 27/3804; B29C 64/106; A61L 27/3683

(57) ABSTRACT

A modified extracellular matrix-based hydrogel according to an example of the present disclosure includes an extracellular matrix-denatured collagen conjugate formed by a Michael addition reaction between an extracellular matrix having an amine group and a denatured collagen into which an ethylenically unsaturated bond functional group is introduced. The modified extracellular matrix-based hydrogel according to the present disclosure exhibits enhanced mechanical properties (e.g., viscoelasticity) compared to the extracellular matrix hydrogel before modification. In addition, it shows a high cell viability when the bioink is prepared by encapsulating cells in a modified extracellular matrix-based hydrogel according to the present disclosure. In addition, when an artificial living tissue for transplantation (for example, artificial corneal tissue) manufactured by 3-D printing bioink according to the present disclosure is transplanted into a damaged cornea, it can be sutured and has a transparency similar to that of the real cornea, and corneal tissue can be reconstructed without other side effects due to its enhanced mechanical properties. Accordingly, the modified extracellular matrix-based hydrogel according to the present disclosure can be applied in tissue engineering fields and related fields requiring improvement in physical properties and is particularly useful as a material for corneal transplants.

### CROSS-REFERENCE TO RELATED APPLICATIONS

This application is based on and claims priority from Korean Patent Application No. 10-2021-0005008, filed on Jan. 14, 2021, with the Korean Intellectual Property Office, the disclosure of which is incorporated herein in its entirety by reference.

### TECHNICAL FIELD

The present disclosure relates to an extracellular matrix-based hydrogel and the like, and more particularly, to an extracellular matrix-based hydrogel modified to enhance mechanical properties such as viscoelasticity, a manufacturing method of the same, and use of the same.

### BACKGROUND

Tissue engineering is a general term for research that aims to regenerate various tissues, and furthermore, to restore organs by appropriately using (stem) cells, a support to which cells are attached to grow, and various factors that can control the growth and differentiation of cells. The field of tissue engineering relies on the use of porous 3-D scaffolds to provide an appropriate environment for tissue and organ regeneration, and the many substances constituting the scaffolds (natural and synthetic materials, biodegradable and permanent materials, etc.) have also been studied. In the early days of tissue engineering, scaffolds made of polymers were manufactured to replace tissues/organs, but recently, a method has been developed in which living cells are directly encapsulated in a scaffold and delivered in vivo. At this time, the material encapsulating the cells is mainly a hydrogel containing a lot of moisture such as collagen, and alginate. However, the hydrogel itself has weak mechanical properties, so it is difficult to be delivered into the human body and replace the actual tissue/organ. Accordingly, many researchers have been studying ways to enhance the physical properties of hydrogels and hydrogel-based scaffolds in various ways such as the preparation of a hybrid type scaffold in which a polymer and a hydrogel are mixed, or a mixture of additional materials such as a hydrogel photocuring agent and a photoinitiator.

For example, Korean Patent No. 10-2146682 discloses a hybrid bioink comprising a first bioink obtained by liquefying the extracellular matrix of decellularized tissue; and a second bioink containing alginate or fibrinogen, in which the first bioink and the second bioink are mixed in a volume ratio (v/v) of 1.5 to 9:1, in which the first bioink is liquefied by digesting the extracellular matrix of the decellularized tissue by pepsin at pH 1 to 3 and a temperature of 15 to 25° C., and in which the first bioink and the second bioink have a concentration of 1.5 to 4.0% (wt/v), respectively. In addition, Korean Patent Laid-Open Patent Publication No. 10-2019-0070922 discloses a bioink including non-denatured neutralized collagen and a crosslinking agent at a concentration greater than 1 mg/ml. In addition, Korean Patent Application Laid-Open No. 2020-0132741 discloses a two-component type bioink composition comprising a first liquid containing methacrylated gelatin and fibrinogen as structural materials, methacrylated low molecular weight collagen as cell transport materials, viscosity enhancers, and lubricants; and a second liquid containing thrombin. In addition, Korean Patent Laid-Open Publication No. 2020-0066218 discloses a bioink composition for 3-D printing comprising a component derived from a microparticulate human tissue and a biocompatible polymer.

### SUMMARY

The present disclosure is derived from the prior technical background, and an object of the present disclosure is to provide a hydrogel and a method for preparing the same to enhance the mechanical properties such as viscoelasticity, etc. and to have excellent biocompatibility, which is beneficial for the survival of cells and can be used for reconstruction of damaged tissues due to small changes in physical properties even after transplantation.

Further, another object of the present disclosure is to provide bioink, artificial living tissue for transplantation, etc., as various uses of the novel hydrogel.

The inventors of the present disclosure create an extracellular matrix-denatured collagen conjugate by combining the extracellular matrix contained in the hydrogel with denatured collagen into which methacryl groups are introduced through a Michael addition reaction to enhance the mechanical properties of the extracellular matrix hydrogel, and the physical properties of the hydrogel containing the extracellular matrix-denatured collagen conjugate and the properties as an artificial tissue material were evaluated. As a result, the hydrogel containing the extracellular matrix-denatured collagen conjugate had enhanced viscoelasticity and showed a high cell viability when the cells were encapsulated using the hydrogel containing the extracellular matrix-denatured collagen conjugate. Further, the present inventors have produced a bioink by encapsulating corneal stromal cells in a hydrogel containing an extracellular matrix-denatured collagen conjugate, prepared an artificial corneal tissue from the bioink, and have confirmed that the artificial corneal tissue could be sutured after transplantation and have transparency similar to the actual cornea, and the corneal tissue could be reconstructed without other side effects, thereby completing the present disclosure.

In order to achieve the above object, an example of the present disclosure provides a modified extracellular matrix-based hydrogel including an extracellular matrix-denatured collagen conjugate formed by a Michael addition reaction between an extracellular matrix having an amine group and a denatured collagen into which an ethylenically unsaturated bond functional group is introduced.

As used in the present disclosure, the term “extracellular matrix (ECM)” is an extracellular part of animal tissue that normally provides structural support to animal cells while performing a variety of other important functions. The extracellular matrix is a term to define connective tissue in animals and consists of various types of proteins including collagen, glycosaminoglycan (GAG), and the like. This extracellular matrix can be tissues of animals such as pigs and cattle and can be extracted from various organs. The corneal-derived extracellular matrix is preferably derived from corneal stromal tissue. In the modified extracellular matrix-based hydrogel according to an example of the present disclosure, the extracellular matrix is preferably decellularized extracellular matrix in consideration of the use of a living body transplant. Accordingly, cells that can act as antigens inducing an immune response are removed to have an effect of minimizing the immune response during allograft or xenograft. Since the type and number of cells and the physical properties of the tissue itself are different depending on the tissue, decellularization is performed using various chemicals such as acids, bases, hypotonic solutions, hypertonic solutions, and detergents. In addition, the extracellular matrix is used while maintaining the structure of the tissue itself through only the decellularization process, but it is used after it is freeze-dried and pulverized, then dissolved in an acidic solution and neutralized again to form a hydrogel. Further, the extracellular matrix preferably is the corneal-derived decellularized extracellular matrix in consideration of mainly applied uses. The corneal-derived decellularized extracellular matrix is derived from the corneal stromal tissue, and includes proteins that help cell adhesion in addition to the physical structure surrounding the cells or proteins that help cell growth and expression of functions. The corneal-derived decellularized extracellular matrix preferably includes collagen fibers from which telopeptide has been removed.

In the modified extracellular matrix-based hydrogel according to an example of the present disclosure, as long as the ethylenically unsaturated bond functional group introduced into the denatured collagen may undergo a Michael addition reaction with an amine group present in the extracellular matrix, the type is largely not limited thereto, and for example, may be selected from the group consisting of a vinyl group, an allyl group, an acryl group, a methacryl group, and the like. In the modified extracellular matrix-based hydrogel according to an example of the present disclosure, the denatured collagen may be preferably selected from methacrylated collagen or acrylated collagen. The methacrylated collagen is a denatured collagen in which a methacryl group is linked to an amine group present in collagen through a peptide bond, and the acrylated collagen is a denatured collagen in which an acryl group is linked to an amine group present in collagen through a peptide bond. The structure and manufacturing method of the methacrylated collagen or acrylated collagen is disclosed in various documents (e.g., U.S. Pat. No. 8,658,711 or He Liang et al., Journal of Materials Chemistry B, 2018, 6, 3703-3715, etc.).

In the modified extracellular matrix-based hydrogel according to an example of the present disclosure, the ethylenically unsaturated bond functional group introduced into the denatured collagen is connected to an amine group present in the extracellular matrix as a single bond through a Michael addition reaction to produce the extracellular matrix-denatured collagen conjugate.

In the modified extracellular matrix-based hydrogel according to an example of the present disclosure, when considering the optimal conditions of the Michael addition reaction, the weight ratio of the extracellular matrix to the denatured collagen contained in the hydrogel is 1:0.05 to 1:0.8, preferably 1:0.1 to 1:0.7, more preferably 1:0.2 to 1:0.6.

The modified extracellular matrix-based hydrogel according to an example of the present disclosure preferably has a non-Newtonian viscosity and has a flow characteristic of shear thinning.

In order to achieve the above object, an example of the present disclosure provides a method for manufacturing modified extracellular matrix-based hydrogel, the method including steps of: preparing an extracellular matrix hydrogel having a pH 2 to 5 by dissolving the extracellular matrix having an amine group in an acid solution; forming an extracellular matrix-denatured collagen conjugate by adding a denatured collagen to which an ethylenically unsaturated bond functional group is introduced to the extracellular matrix hydrogel, mixing them uniformly, and inducing a Michael addition reaction; and neutralizing the hydrogel including the extracellular matrix-denatured collagen conjugate to a pH 5.5 to 8. In the method for manufacturing a modified extracellular matrix-based hydrogel according to an example of the present disclosure, referring to the above description, detailed description thereof is excluded for the technical characteristics of an extracellular matrix having an amine group, denatured collagen introduced with an ethylenically unsaturated bond functional group, Michael addition reaction, and extracellular matrix-denatured collagen conjugate, etc.

In the method for manufacturing a modified extracellular matrix-based hydrogel according to an example of the present disclosure, the type of acid solution used for dissolving extracellular matrix is not particularly limited, and a weak acid solution is preferable in consideration of the use for living transplantation. The weak acid may be selected from the group consisting of acetic acid, citric acid, butyric acid, palmitic acid, oxalic acid, tartaric acid, malic acid, succinic acid, and the like. In addition, the pH of the extracellular matrix hydrogel is preferably 2.5 to 4.5 in consideration of the optimal conditions for dissolution of the extracellular matrix or the optimal conditions for the Michael addition reaction. Further, the amount of extracellular matrix in the extracellular matrix hydrogel is not significantly limited, and it is preferably 1 to 4% (w/v), more preferably 1.5 to 3% (w/v) in consideration of the optimal conditions for dissolution of extracellular matrix, the optimal conditions for Michael addition reaction, or the like.

In the manufacturing method of the modified extracellular matrix-based hydrogel according to an example of the present disclosure, the amount of the denatured collagen added is preferably 5 to 80 parts by weight, more preferably 10 to 70 parts by weight, most preferably 20 to 60 parts by weight based on 100 parts by weight of extracellular matrix contained in the hydrogel in consideration of the optimal condition of the Michael addition reaction.

In the method for manufacturing a modified extracellular matrix-based hydrogel according to an example of the present disclosure, the temperature condition of the Michael addition reaction is preferably 0 to 15° C., more preferably 1 to 10° C. in consideration of the denaturation prevention or reaction efficiency of the hydrogel. In addition, the time condition of the Michael addition reaction is preferably 5 to 30 hours, more preferably 10 to 20 hours in consideration of reaction efficiency and the like.

In the method for manufacturing a modified extracellular matrix-based hydrogel according to an example of the present disclosure, the hydrogel including the extracellular matrix-denatured collagen conjugate is preferably neutralized to a pH 6.1 to 7.6 in consideration of the use of a biotransplantation.

In order to achieve the above object, an example of the present disclosure provides a bioink consisting of or including the modified extracellular matrix-based hydrogel described above. The bioink according to a preferred example of the present disclosure has a form of a composition including cells and the above-described modified extracellular matrix-based hydrogel, and the cells are present in a form encapsulated in the modified extracellular matrix-based hydrogel.

The term “bioink” as used in the present disclosure is defined as a cell compatible material capable of 3-D printing. The bioink can be extruded through a needle at 0 to 37° C., and then it can be gelled or solidified. The bioink can be formulated to be suitable for inkjet, laser-assisted, or microvalve 3-D printing equipment.

The concentration of cells in the bioink according to a preferred example of the present disclosure is not significantly limited, and is preferably 1×10⁶ cells/ml to 1×10⁷ cells/ml, and more preferably 3×10⁶ cells/ml to 8×10⁶ cells/ml in consideration of the ease of molding by 3-D printing, the effect of tissue reconstruction after transplantation, etc.

In the bioink according to a preferred example of the present disclosure, the cells may be selected from cells derived from various tissues and are preferably corneal-derived cells in consideration of their main application. The corneal-derived cells may be one or more selected from the group consisting of corneal endothelial cells, corneal epithelial cells, and corneal stromal cells, preferably a double corneal stromal cell.

In order to achieve the above object, an example of the present disclosure provides an artificial living tissue for transplantation, molded from the aforementioned bioink by 3-D printing. The artificial living tissue for transplantation has various types or ranges of transplanted living tissue depending on the origin of the modified extracellular matrix-based hydrogel constituting the bioink or cells, and is preferably an artificial living tissue for transplantation, which is transplanted to the damaged cornea in consideration of the tissue reconstruction effect.

The modified extracellular matrix-based hydrogel according to the present disclosure exhibits enhanced mechanical properties (e.g., viscoelasticity) compared to the extracellular matrix hydrogel before modification. In addition, it shows a high cell viability when the bioink is prepared by encapsulating cells in a modified extracellular matrix-based hydrogel according to the present disclosure. In addition, when an artificial living tissue for transplantation (for example, artificial corneal tissue) manufactured by 3-D printing bioink according to the present disclosure is transplanted into a damaged cornea, it can be sutured and has a transparency similar to that of the real cornea, and corneal tissue may be reconstructed without other side effects due to its enhanced mechanical properties. Accordingly, the modified extracellular matrix-based hydrogel according to the present disclosure may be applied in tissue engineering fields and related fields requiring improvement in physical properties and is particularly useful as a material for corneal transplants.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows the results of FT-IR analysis of the modified corneal-derived decellularized extracellular matrix hydrogel prepared in an embodiment of the present disclosure.

FIG. 2 shows the results of analyzing the change in viscoelasticity of the modified corneal-derived decellularized extracellular matrix hydrogel prepared in an embodiment of the present disclosure.

FIG. 3 shows the results of measuring the cell viability in the bioink prepared in an embodiment of the present disclosure with an optical microscope.

FIG. 4 shows a slit lamp image and an optical coherence tomography (OCT) image among the in-vivo evaluation results performed in an embodiment of the present disclosure.

FIG. 5 is a graph showing a behavioral optometry evaluation result among in-vivo evaluation results performed in an embodiment of the present disclosure.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, the present disclosure is described in detail through embodiments. However, the following embodiments are only for clearly illustrating the technical characteristics of the present disclosure, and do not limit the scope of protection of the present disclosure.

### 1. Preparation of Corneal-Derived Decellularized Extracellular Matrix (Co-dECM) and Hydrogel Containing the Same

Corneal-derived decellularized extracellular matrix (Co-dECM) was prepared as follows (See. Kim H, Park M N, Kim J, Jang J, Kim H K and Cho D W 2019 Characterization of cornea-specific bioink: high transparency, improved in vivo safety J. Tissue Eng. 10). First, the entire cornea incised from the calf s eye was washed with a PBS buffer solution containing 100 units/ml of penicillin and 0.1 mg/ml of streptomycin. Thereafter, the epithelium and endothelium were removed from the corneal tissue to obtain a pure corneal stromal layer. Thereafter, the matrix tissue was placed in a 20 mM ammonium hydroxide solution (NH₄OH; 4.98 N aqueous solution) containing 0.5% Triton X-100, and the mixture was stirred for about 4 hours. Thereafter, the matrix tissue was washed with distilled water and treated with a hypotonic Tris hydrochloride (Tris-HCl; pH 7.4) buffer solution for about 24 hours. Thereafter, the matrix tissue was placed in a 10 mM Tris-HCl solution containing 1% (v/v) Triton X-100, and the mixture was stirred at 37° C. for about 24 hours to obtain a corneal-derived decellularized extracellular matrix (Co-dECM) tissue. Thereafter, the corneal decellularized extracellular matrix (Co-dECM) tissue was sterilized by treatment with 1% peracetic acid solution in 50% ethanol for about 10 hours. After completion of the decellularization process, corneal-derived decellularized extracellular matrix (Co-dECM) was freeze-dried overnight and ground into a fine powder using liquid nitrogen and a grinding device. 0.2 g Co-dECM powder was added to 10 ml of acetic acid solution (0.5 M) supplemented with 0.02 g pepsin, and the mixture was uniformly stirred through vortexing, and treated for about 3 days to remove telopeptides in collagen molecules and be completely dissolved to obtain a corneal-derived decellularized extracellular matrix hydrogel having a pH of about 3 to 4 and a corneal-derived decellularized extracellular matrix (Co-dECM) concentration of 2% (w/v). The 2% (w/v) Co-dECM hydrogel was filtered through a 10 μm mesh and stored at 4° C., and it was used for subsequent experiments.

### 2. Preparation of Modified Corneal-Derived Decellularized Extracellular Matrix (Co-dECM)-Based Hydrogel Using Michael Addition Reaction

### Modified Hydrogel Preparation Example 1

0.05 g of methacrylated collagen (MAC) was added to 10 ml of corneal-derived decellularized extracellular matrix hydrogel, and the mixture was uniformly stirred through vortexing. While stored at 4° C. for 16 hours, a Michael addition reaction was induced between the methacrylated collagen and the conical-derived decellularized extracellular matrix to modify the corneal-derived decellularized extracellular matrix. Thereafter, a 10 N sodium hydroxide solution was added to the hydrogel to be neutralized to be pH about 7.0 to 7.4 on ice, thereby obtaining a modified corneal-derived decellularized extracellular matrix-based hydrogel.

### Modified Hydrogel Preparation Example 2

Except that 0.01 g of methacrylated collagen (MAC) was added to 10 ml of corneal-derived decellularized extracellular matrix hydrogel, the modified corneal-derived extracellular matrix-based hydrogel was obtained under the same conditions and in the same manner as in Preparation Example 1.

### Modified Hydrogel Preparation Example 3

Except that 0.1 g of methacrylated collagen (MAC) was added to 10 ml of corneal-derived decellularized extracellular matrix hydrogel, the modified corneal-derived extracellular matrix-based hydrogel was obtained under the same conditions and in the same manner as in Preparation Example 1.

### 3. Characterization of Modified Hydrogels

### (1) Analysis of Chemical Changes in Modified Hydrogels

Chemical changes of the modified corneal-derived decellularized extracellular matrix-based hydrogel prepared in Modified Hydrogel Preparation Examples 1 to 3 were measured through FT-IR analysis.

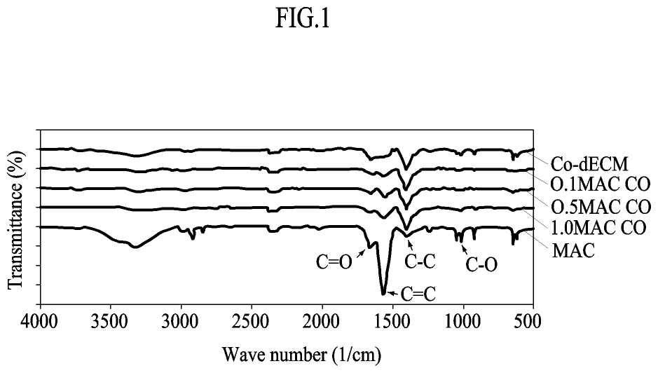

FIG. 1 shows the results of FT-IR analysis of the modified corneal-derived decellularized extracellular matrix hydrogel prepared in an embodiment of the present disclosure. In FIG. 1, the term “Co-dECM” refers to corneal-derived decellularized extracellular matrix hydrogel before modification, and “MAC” refers to methacrylated collagen. In addition, in FIG. 1, the term “0.5MACCO” refers to the modified corneal-derived decellularized extracellular matrix-based hydrogel prepared in Modified Hydrogel Preparation Example 1, and the term “0.1MACCO” refers to the modified corneal-derived decellularized extracellular matrix-based hydrogel prepared in Modified Hydrogel Preparation Example 2, and the term “1.0MACCO” refers to the modified corneal-derived decellularized extracellular matrix-based hydrogel prepared in Modified Hydrogel Preparation Example 3.

In addition, the values of peaks corresponding to C—C single bonds and C═C double bonds among the FT-IR analysis results of the modified corneal-derived decellularized extracellular matrix hydrogel prepared in embodiments of the present disclosure are summarized in Table 1 below.

TABLE 1

| Classi- | | | | | |
|---|---|---|---|---|---|
| fication | MAC | 1.0MACCO | 0.5MACCO | 0.1MACCO | Co-dECM |
| C═C | −17.47 | −2.92 | −2.40 | −1.87 | −1.46 |
| peak | | | | | |
| value | | | | | |
| C—C | −2.44 | −5.81 | −5.83 | −5.42 | −5.06 |
| peak | | | | | |
| value | | | | | |

In the modified hydrogel Preparation Examples 1 to 3, the Michael addition reaction between the methacrylated collagen and the corneal-derived decellularized extracellular matrix proceeds by the following mechanism.

### Mechanism of Michael Addition Reaction Between Methacrylated Collagen and Corneal-Derived Decellularized Extracellular Matrix

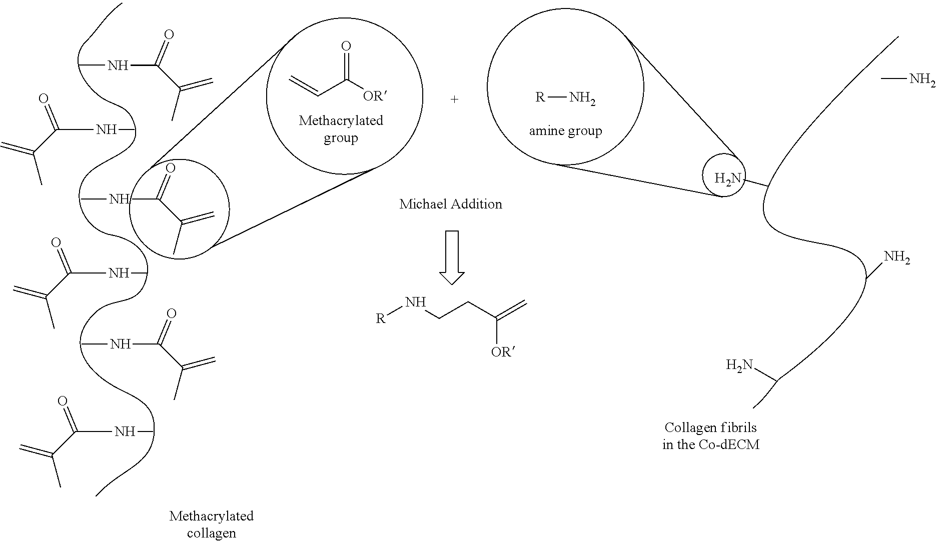

In the corneal-derived decellularized extracellular matrix hydrogel having a pH of about 3 to 4, the amine group present in the corneal-derived decellularized extracellular matrix has high reactivity to lose protons easily, and ethylenically unsaturated bonds present in methacrylated collagen obtains a proton source. Thereafter, it forms a single bond with an amine group present in the corneal-derived decellularized extracellular matrix.

0.1% (w/v), 0.5% (w/v) and 1.0% (w/v) methacrylated collagens (MAC) were added to corneal-derived decellularized extracellular matrix (Co-dECM) hydrogel, respectively, and Michael addition reaction was induced to change the amount of C—C single bond and C═C double bond. As shown in FIG. 1 and Table 1, methacrylated collagen (MAC) contains methacrylic groups and thus has the most C═C double bonds, whereas corneal-derived decellularized extracellular matrix (Co-dECM) had the fewest C═C double bonds. When methacrylated collagen (MAC) reacts with corneal-derived decellularized extracellular matrix (Co-dECM), the C═C double bond is reduced, and C—C single bond is increased compared to methacrylated collagen (MAC), indicating Michael addition reaction proceeded.

In the case of 0.1MACCO hydrogel, C—C single bonds and C═C double bonds were increased compared to Co-dECM hydrogel. In the case of 0.5MACCO hydrogel, C—C single bonds and C═C double bonds were increased compared to Co-dECM hydrogel, and C—C single bonds and C═C double bonds were also increased compared to 0.1MACCO hydrogel. These results indicate that the reaction between Co-dECM and MAC proceeded more in the case of the 0.5MACCO hydrogel compared to the 0.1MACCO hydrogel. Meanwhile, in the case of 1.0MACCO hydrogel, C—C single bonds and C═C double bonds were increased compared to Co-dECM hydrogel, but C—C single bonds were almost the same compared to 0.5MACCO hydrogel. These results indicate that the reaction is closest to the maximum in the 0.5 MACCO hydrogel.

From the results of FIG. 1 and Table 1, the present inventors found that the concentration of methacrylated collagen (MAC) for the modification of corneal-derived decellularized extracellular matrix (Co-dECM) hydrogel was 0.5% (w/v), and the modified corneal-derived decellularized extracellular matrix-based hydrogel prepared in Modified Hydrogel Preparation Example 1 was used in the subsequent preparation experiments of bioink and artificial corneal tissue for transplantation.

### (2) Analysis of Changes in Viscoelasticity of Modified Hydrogels

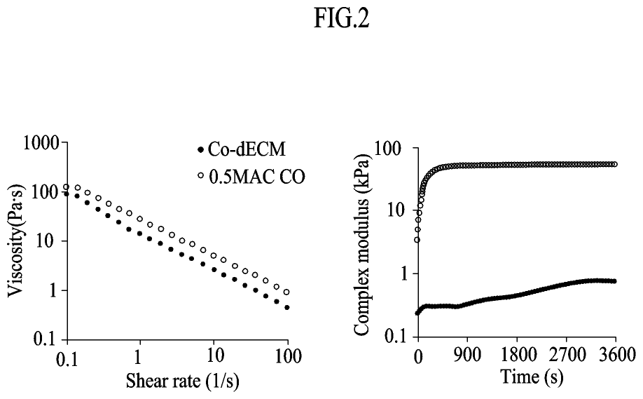

In order to analyze the change in viscoelasticity of the modified hydrogel, the rheological properties were measured using an advanced hybrid rheometer equipped with a 25 mm diameter plate. First, the normal shear sweep analysis of the hydrogel was performed at 4° C., a temperature condition generally used in the biofabrication process, and specifically, the viscosity according to the shear rate was measured. Further, the time sweep analysis of the hydrogel was performed at 37° C., a temperature condition used after the biofabrication process, and specifically, the complex modulus (G*) at 2% strain over time was measured. The time sweep analysis is used to study the gelation kinetics of hydrogels. FIG. 2 shows the results of analyzing the change in viscoelasticity of the modified corneal-derived decellularized extracellular matrix hydrogel prepared in an embodiment of the present disclosure. The graph on the left of FIG. 2 shows a normal shear sweep analysis result, and the graph on the right shows a time sweep analysis. In addition, in FIG. 2, the term “Co-dECM” refers to the corneal-derived decellularized extracellular matrix hydrogel before modification, and “0.5MACCO” refers to the modified corneal-derived extracellular matrix-based hydrogel prepared in Modified Hydrogel Preparation Example 1. As shown in the normal shear sweep analysis result of FIG. 2, it was confirmed that both the corneal-derived decellularized extracellular matrix hydrogel before modification and the modified corneal-derived extracellular matrix-based hydrogel prepared in Preparation Example 1 have shear thinning properties, and there was no significant difference between the two materials. However, as shown in the time sweep analysis result of FIG. 2, the complex modulus (G*) value of the modified corneal-derived extracellular matrix-based hydrogel prepared in Preparation Example 1 was approximately 78 times greater than the corneal-derived decellularized extracellular matrix hydrogel before modification after about 30 minutes. This result means that the effect of the Michael addition reaction appears in the gelation process of the hydrogel.

### 4. Preparation of Bioink and Artificial Corneal Tissue for Transplantation

### Bioink Preparation Example 1

A bioink was prepared by encapsulating the differentiated keratocytes in the modified corneal-derived decellularized extracellular matrix-based hydrogel prepared in Preparation Example 1.

Differentiated keratocytes were prepared as follows (See. [Park M N, Kim B, Kim H, Park S H, Lim M H, Choi Y J, Yi H G, Jang J, Kim S W and Cho D W 2017 Human turbinate-derived mesenchymal stem cells differentiated into keratocyte progenitor cells J. Clin. Exp. Ophthalmol. 8 627]. Human turbinate derived mesenchymal stem cells (hTMSCs; obtained from Catholic University of Korea, St. Mary's Hospital) were placed in normal DMEM (Dulbecco's Modified Eagle's Medium) containing 10% (v/v) fetal bovine serum and 1% (v/v) penicillin and cultured in a humidified 5% carbon dioxide atmosphere and a temperature of 37° C. Then, in the second passage, the normal medium was replaced with a differentiation medium containing 10 ng/ml KGF/EGF and cultured for one day to obtain differentiated keratocytes. Thereafter, the keratocytes obtained from the second or third passage were encapsulated at a concentration of 5×10⁶ cells/ml in the modified corneal-derived extracellular matrix-based hydrogel prepared in Preparation Example 1 to prepare the bioink in the form of the corneal decellularized extracellular matrix (Co-dECM) hydrogel in which the cells were encapsulated. The cell encapsulation process was performed on ice. In addition, an artificial corneal tissue having a diameter of 4 mm and a height of 200 μm was produced from the bioink using a 3-dimensional cell printing system.

### Bioink Preparation Example 2

Except that differentiated keratocytes were encapsulated in a corneal-derived decellularized extracellular matrix hydrogel before modification, a bioink was prepared, and an artificial corneal tissue was produced under the same conditions and in the same manner as in Bioink Preparation Example 1.

### 5. Cell Viability Analysis

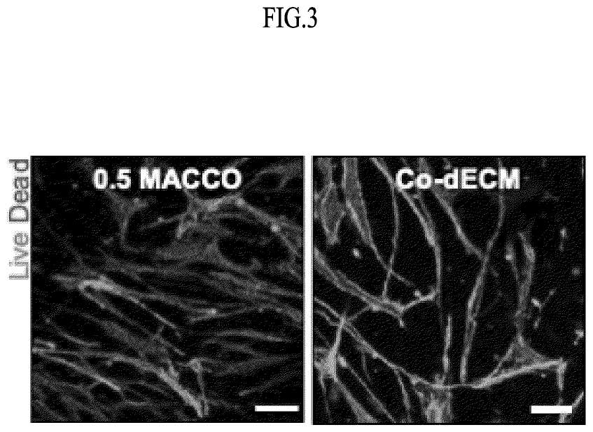

FIG. 3 shows the results of measuring the cell viability in the bioink prepared in an embodiment of the present disclosure with an optical microscope. In FIG. 3, the term “0.5MACCO” refers to the bioink prepared in Bioink Preparation Example 1, and the term “Co-dECM” refers to the bioink prepared in Bioink Preparation Example 2. As shown in FIG. 3, both the case of using the modified corneal-derived extracellular matrix-based hydrogel prepared in Preparation Example 1 as a hydrogel for encapsulating cells and the case of using the corneal-derived decellularized extracellular matrix hydrogel before modification showed 95% or more cell viability.

### 6. In-vivo Evaluation

In order to observe the in-vivo compatibility and the level of vision recovery after transplantation of the artificial corneal tissue prepared in Bioink Preparation Example 1, animal experiments were performed in Daegu-Gyeongbuk Advanced Medical Industry Promotion Foundation (DGMIF, the approval number of protocol: DGMIF-17080801-00) according to the ARVO statement on the use of animals by Ophthalmic and Vision Research. Eight healthy beagle dogs (8 weeks old male having an average weight of about 4 kg) were anesthetized with 30 mg/ml of ketamine and 10 mg/ml of rompun. A 3-quarter annular incision with a diameter of 5 mm was made using a crescent knife. Thereafter, the beagles with corneal scratches were divided into two groups, such as a negative control group and an experimental group. No separate treatments were taken for the beagle corresponding to the negative control group. The artificial corneal tissue prepared in Bioink Preparation Example 1 was transplanted on the corneal matrix of the beagles corresponding to the experimental group. Meanwhile, in the case of transplanting the artificial corneal tissue prepared in Bioink Preparation Example 2, the incision site was not smoothly sutured as a result of the pre-test, so it was not included in the experimental group. Then, the incision site was sutured with 10-0 ethilon nylond, and the operated eye was treated with eye drops containing olymyxin B, neomycin and dexamethasone (Forus, Samil Pharm. Co., Ltd, Korea) once a day for 2 weeks.

For the experimental animals, slit lamp examination and OCT examination were performed at predetermined time intervals for 24 weeks after transplantation. Further, behavioral optometry was evaluated in a state in which only the incised eye was exposed for visual acuity test 5 weeks after transplantation of the experimental animals. The behavioral optometry evaluation was performed by the veterinarian, which is one of the animal visual acuity evaluations that indirectly evaluate visual acuity by evaluating whether it avoids obstacles and whether it can follow moving objects with its eyes, with a full score of 5.

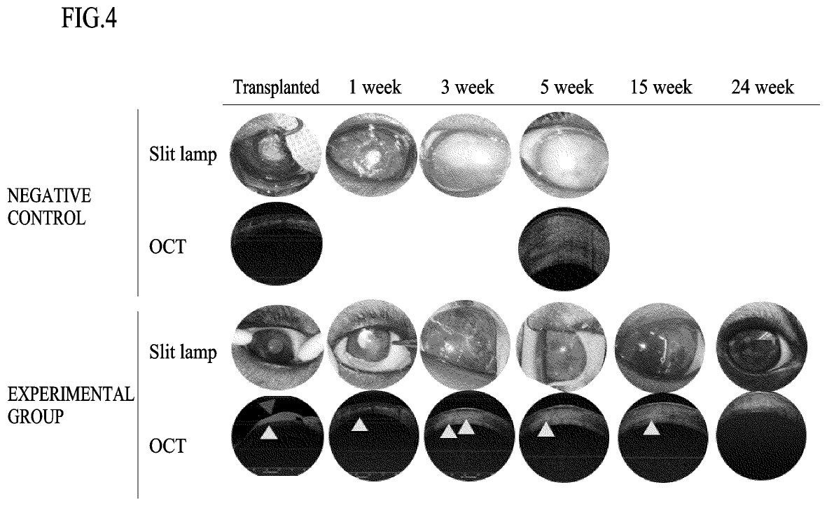

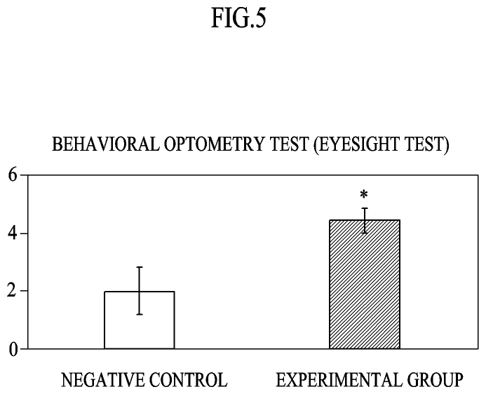

FIG. 4 shows a slit lamp image and an optical coherence tomography (OCT) image among the in-vivo evaluation results performed in an embodiment of the present disclosure. As shown in FIG. 4, in the case of the negative control group, as time passed, corneal edema occurred entirely in addition to the scratched area in the cornea, and the cornea became cloudy. On the other hand, in the case of the experimental group, not only could the cornea be sutured, but the transparency gradually recovered as time passed. FIG. 5 is a graph showing a behavioral optometry evaluation result among in-vivo evaluation results performed in an embodiment of the present disclosure. As shown in FIG. 5, the negative control group showed a significant decrease in visual acuity, while the experimental group recorded a value close to 5, the perfect score for behavioral optometry, indicating that visual acuity was greatly recovered. These results indicate that in the case of the artificial corneal tissue prepared in Bioink Preparation Example 1, it is possible to suture the incised cornea and greatly help in corneal reconstruction, such as tolerating internal intraocular pressure well and recovering visual acuity even after transplantation.

As described above, the present disclosure has been described through the above embodiments, but the protection scope of the present disclosure is not necessarily limited thereto, and various modifications can be made within the scope and spirit of the present disclosure. Accordingly, the protection scope of the present disclosure should not be limited to the specific embodiments disclosed as the best mode but should be construed to include all embodiments falling within the scope of the claims appended to the present disclosure.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A modified extracellular matrix-based hydrogel comprising an extracellular matrix-denatured collagen conjugate formed by a Michael addition reaction between the extracellular matrix having an amine group and denatured collagen to which an ethylenically unsaturated bond functional group is introduced.

2. The modified extracellular matrix-based hydrogel of claim 1, wherein the extracellular matrix is decellularized extracellular matrix.

3. The modified extracellular matrix-based hydrogel of claim 2, wherein the decellularized extracellular matrix is corneal-derived decellularized extracellular matrix.

4. The modified extracellular matrix-based hydrogel of claim 1, wherein the ethylenically unsaturated bond functional group is selected from the group consisting of a vinyl group, an acryl group and a methacryl group.

5. The modified extracellular matrix-based hydrogel of claim 1, wherein the denatured collagen is selected from methacrylated collagen or acrylated collagen.

6. The modified extracellular matrix-based hydrogel of claim 1, wherein a weight ratio of the extracellular matrix to the denatured collagen contained in the hydrogel is 1:0.05 to 1:0.8.

7. A method for manufacturing the modified extracellular matrix-based hydrogel of claim 1, the method comprising steps of:
preparing an extracellular matrix hydrogel having a pH 2 to 5 by dissolving the extracellular matrix having an amine group in an acid solution;
forming an extracellular matrix-denatured collagen conjugate by adding a denatured collagen to which an ethylenically unsaturated bond functional group is introduced to the extracellular matrix hydrogel, mixing them uniformly, and inducing a Michael addition reaction; and
neutralizing the hydrogel including the extracellular matrix-denatured collagen conjugate to a pH 5.5 to 8.

8. The method for manufacturing modified extracellular matrix-based hydrogel of claim 7, wherein the extracellular matrix is corneal-derived decellularized extracellular matrix.

9. The method for manufacturing modified extracellular matrix-based hydrogel of claim 7, wherein the denatured collagen is selected from methacrylated collagen or acrylated collagen.

10. The method for manufacturing modified extracellular matrix-based hydrogel of claim 7, wherein an addition amount of the denatured collagen is 5 to 80 parts by weight relative to 100 parts by weight of the extracellular matrix contained in the hydrogel.

11. The method for manufacturing modified extracellular matrix-based hydrogel of claim 7, wherein an amount of the extracellular matrix in the extracellular matrix hydrogel is 1 to 4% (w/v).

12. A bioink comprising cells and the modified extracellular matrix-based hydrogel of claim 1, wherein the cells are present in an encapsulated form in the modified extracellular matrix-based hydrogel.

13. The bioink of claim 12, wherein a concentration of cells in the bioink is 1×10⁶ cells/ml to 1×10⁷ cells/ml.

14. The bioink of claim 12, wherein the cells are corneal-derived cells.

15. An artificial living tissue for transplantation, molded from the bioink of claim 12 by 3-D printing.
